(19) United States
(12) Patent Application Publication
(10) Pub. No.: US 2005/0008763 A1
(43) Pub. Date: Jan. 13, 2005

(54) Antimicrobial coatings for medical applications

(76) Inventor: Steven C. Schachter, Sharon, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/668,681

(22) Filed: Sep. 23, 2003

Related U.S. Application Data
(60) Provisional application No. 60/413,115, filed on Sep. 24, 2002.

(51) Int. Cl. A61K 9/00

(52) U.S. Cl. 427/2.24

(57) ABSTRACT

The invention generally is related to preventing or inhibiting microbial infections on live tissues or in relation to the use of medical articles. In one embodiment, the invention is directed to a method for forming an antimicrobial coating on live tissue or a medical article, such as, for example, a catheter. The method includes applying to a live tissue or a surface of a medical article an aqueous or non-aqueous composition that includes at least one silane of the general formula R¹ₙSi(OR²)₄₋ₙ wherein, n is an interger of 1 or 2, R¹ generally is a lower alkyl group, a C₆-C₈ aryl group, or a functional group, such as vinyl, acrylic, amino, mercapto, or vinyl chloride functional group, and R² generally is a lower alkyl group. A partial condensate of a silanol of the formula R¹Si(OH)₃ also can be used. The composition includes one or more additional ingredients. The method also includes reacting and/or curing the silane, in the presence of the one or more ingredients, thereby forming the antimicrobial coating.

### RELATED APPLICATION

This application claims the benefit of U.S. Provisional Application No. 60/413,115, filed Sep. 24, 2002, the teachings of which are incorporated herein by reference in their entirety.

### BACKGROUND OF THE INVENTION

The use of invasive devices, such as intravascular devices (catheters), is often complicated by a local or systemic infections including septic thrombophlebitis, endocarditis, bloodstream infections, and metastatic infection (e.g., osteomyelitis, endophthalmitis, arthritis) resulting from hematogenous seeding of another body site by a colonized catheter.

Catheter-related infections, particularly catheter-related bloodstream infections, are associated with increased morbidity, mortality rates of 10% to 20%, prolonged hospitalization (mean of 7 days) and increased medical costs, in excess of $6,000 (1988 dollars) per hopsitalization. Thus there is a need for measures that reduce the risk of intravascular-related infections.

Infections also complicate the chronic use of invasive devices, such as bladder catheters, adding to the morbidity and costs associated with their use, which commonly occurs in hospitals and chronic care facilities.

While the pathogenesis of catheter-related infections is multifactorial, most infections appear to result from migration of skin organisms at the insertion site into the cutaneous catheter tract with eventual colonization of the catheter trip. Other important pathogenic determinants of catheter-related infection include properties of the material used to make the device and the intrinsic properties of the infecting organism. In vitro studies suggest that catheters made of polyvinyl chloride or polyethylene may be less resistant to the adherence of microorganisms that are catheters made of Teflon®, silicone elastomer, or polyurethane. Some catheter materials also have surface irregularities that may further enhance microbial adherence and thus make microbial colonization and subsequent infection more likely to occur.

Therefore, a need continues to exist for methods for preventing or inhibiting microbial infections related to the use of medical articles having surfaces that can be contaminated with microorganisms. A need exists for medical articles that have properties that reduce or eliminate the risk of microbial contamination or colonization and for methods of producing them. In particular, a need exists for catheters that have antiseptic, antimicrobial, or sterile properties and for methods for producing them.

### SUMMARY OF THE INVENTION

The invention generally is related to inhibiting or preventing microbial infections on live tissue, medical devices and other articles used for medical applications.

In one embodiment, microbial infections are inhibited or prevented by a method that includes applying to live tissue or a medical article substrate an aqueous or non-aqueous composition that includes at least one silane of the general formula R¹ₙSi(OR²)₄₋ₙ wherein, n is an interger of 1 or 2, R¹ generally is a lower alkyl group, a C₆-C₈ aryl group, or a functional group, such as vinyl, acrylic, amino, mercapto, or vinyl chloride functional group, and R² generally is a lower alkyl group. A partial condensate of a silanol of the formula R¹Si(OH)₃ also can be used. The composition includes one or more additional ingredients, as described below. The method also includes reacting, e.g., forming a partial silanol condensate, and/or curing the silane, in the presence of the one or more ingredients, thereby forming an antimicrobial coating onto the live tissue or the medical article substrate.

In another embodiment, the invention is directed to a method for producing a medical article, such as, for example, a catheter, that has an antimicrobial coating. The method comprises applying to a surface of a medical device an aqueous or non-aqueous composition that includes at least one silane of the general formula R¹ₙSi(OR²)₄₋ₙ wherein, n is an interger of 1 or 2, R¹ generally is a lower alkyl group, a C₆-C₈ aryl group, or a functional group, such as vinyl, acrylic, amino, mercapto, or vinyl chloride functional group, and R² generally is a lower alkyl group. A partial condensate of a silanol of the formula R¹Si(OH)₃ also can be used. The composition includes one or more additional ingredients, as described below. The method also includes reacting, e.g., forming a partial silanol condensate, and/or curing the silane, in the presence of the one or more ingredients, thereby forming the antimicrobial coating.

In yet another embodiment, the invention is directed to a medical article having an antimicrobial coating produced by applying to a surface (interior and/or exterior) of a medical article, e.g., a device such as a catheter, the composition described above and reacting and/or curing the silane or silanol described above in the presence of one or more additional ingredients.

The invention has many advantages. The aqueous or non-aqueous compositions used in the methods of the invention can be easily prepared and applied, using commercially available compounds. The compositions are believed to penetrate well into surface irregularities and to produce smooth and strongly adherent coatings when applied to medical articles. It is believed that the coatings have antimicrobial properties and have no toxic effects. Furthermore, commercially manufacturing the coated articles generally is expected to generate only a small amount of volatile organic compounds.

Compared to other coatings, such as, for instance, acrylics, polyurethanes, epoxies, or phenolics, the compositions employed to coat a medical article are believed to penetrate into surface irregularities, crevices and inclusions. The compositions are believed to have low cohesive forces and to tend to wick into micro-voids and even into inclusions as small as nanometer size.

### DETAILED DESCRIPTION OF THE INVENTION

A description of preferred embodiments of the invention follows.

In one aspect, the invention is related to preventing or inhibiting microbial infections related to the use of medical articles. Such medical articles either directly contact internal organs, tissues, body cavities and fluids in a human or animal subject or can themselves remain external to the body but can transfer microorganisms to a human or animal subject indirectly, by contact with blood, plasma, oxygen, intravenous or feeding solutions, equipment used in preparation of dental or surgical cements and other media that are supplied to the subject, for instance, during transfusions, artificial breathing or intravenous feeding.

Examples of medical articles include medical devices, in particular invasive medical devices that are used in surgical procedures, medical diagnosis, medical tests, chronic conditions, in veterinary, laboratory animals and other applications. Generally, these articles come in contact with internal organs, tissues, body cavities, e.g., bladder, veins, arteries, or with body fluids. Specific examples include catheters (e.g., bladder, vascular, neurointerventional, angioplasty, urinary and other catheters known in the art), biopsy or amniocentesis needles, stents (e.g., urinary stents), intrauterine devices, shunts, cannulae, lines, tubes (e.g., oxygen, esophageal, gastroenteral, drainage, tracheal tubes), vena cava filters, devices related to ultrasound probes, sensors, wires, (e.g., guidewires, such as pacemaker leads), feeding tubes, heart bypass devices, heart circulation assist devices, proctoscopes and others. Surgical implants, such as, for instance, artificial joints, screws, plates (e.g., skull plates), dental or surgical cement substrates, leads or wires, artificial organs, (e.g., artificial hearts or artificial heart valves), dentures, contact lenses, implanted sensors, and others also can be coated as further described below.

Yet other medical articles that can be coated with antimicrobial coatings, as further described below, include tubes, pouches, containers, valves, filters, surgery and testing equipment and other articles that are external to the body but which, through contact with body fluids (e.g., blood, plasma), intravenous feeding solutions, oxygen and through other means, can transfer infections-causing microorganisms to a human or animal subject.

Medical articles included herein often are fabricated from a polymeric material, e.g., polyvinyl chloride, polyethylene, rubber, Teflon®, silicone elastomers, polyurethane and others. They can include combinations of materials. Medical articles that have metal surfaces also can be used. In other examples, the medical articles have ceramic or cement surfaces.

The medical articles can have any shape or dimensions, as known in the art.

Either or both interior and exterior surfaces of medical articles can be coated by applying an aqueous or non-aqueous composition as further described below. In the case of a catheter, for example, it is preferred to form antimicrobial coatings on both interior and exterior surfaces. Internal tube surfaces are coated for tubing that remains external to the body and is used for transferring fluids to a human or animal subject; coating of external tube surfaces in such cases is optional.

The compositions can be applied by dipping, wiping, painting (e.g., with a brush) spraying or other methods known in the art. Several coats can be overlaid one upon another. Optionally, the surface of the medical article are cleaned and/or pretreated with a primer.

Once applied, components in the aqueous or non-aqueous composition undergo physical and/or chemical changes, e.g., solvent evaporation, crosslinking (curing), thereby forming the antimicrobial coating onto the surface of the medical article. Solvent evaporation and/or curing can occur at ambient condition or can be promoted or facilitated by heating, as is known in the art. The resulting medical articles have an antimicrobial coating.

The medical articles, that include the microbial coatings described herein, can be stored under sterile conditions, as known in the art, thereby reducing and minimizing exposure to microorganisms prior to use.

Preferred coatings are non-toxic and can withstand contact with body fluids or chemical compounds typically used. Also preferred are coatings that have good adherence to the substrate and that do not peel or flake. In some embodiments, the coatings resist wear or cracking upon bending or folding of a flexible substrate. Coatings that are smooth and have reduced porosity are particularly preferred.

The coatings are believed to have antimicrobial properties.

As used herein, the term “antimicrobial” refers to inhibition of, prevention of or protection against microorganisms such as, bacteria, microbes, fungi, viruses, spores, yeasts, molds and others generally associated with infections such as those contracted from the use of the medical articles described herein.

Without wishing to be bound by a particular interpretation of the mechanism by which the coated medical articles described herein protect against infections, it is believed that they are non-adherent towards microorganisms and that their smooth surfaces prevent penetration and propagation of microorganisms. In addition, the coatings are believed to fill surface interstices and thus prevent or minimize microbial colonization.

The aqueous or non-aqueous compositions and the resulting coatings, that are further described below, can include or can be combined with additional antimicrobial, (e.g., antibacterial, antiviral, antifungal, etc.) agents or formulations, such as known in the pharmaceutical arts. In one example, the antimicrobial coatings can further include zinc or other metallic species that impart antimicrobial properties.

In another aspect, the invention is related to preventing or inhibiting microbial infections by applying antimicrobial compositions described herein onto live tissue in a human or animal. Examples include tissues exposed during surgery, implant or diagnostic interventions, for instance during hip replacement, oral or eye surgery, hip/joint replacement and others. Dental tissues, skin, wounds or burns also may be protected against infection with the compositions described herein.

Application can be by coating, spraying or other techniques known in the medical arts. Application and curing are conducted under conditions that do not adversely affect the living tissue. For instance, curing is conducted at ambient temperature or under mild heating to temperatures suitable for living tissue.

When applied and cured onto a region of live tissue, the compositions form an antimicrobial coating onto that tissue region, that inhibits or prevents microbial growth or colonization. In a preferred application, the compositions are applied at the base of teeth in a human or animal subject to form an antimicrobial coating, for example, for the prevention or treatment of gingivitis.

The antimicrobial compositions of the invention that are applied to living tissue can be formulated with additional pharmaceutically suitable excipients, or with other active agents, such as, for example, with additional antimicrobial, analgesic or antiinflammatory agents.

The aqueous or non-aqueous compositions and the resulting antimicrobial coatings include at least one organosilicon compound. Organosilicon compouds are known in the art. Examples include, but are not limited to, silanes, alkyl- or alkoxysilanes, silanols, silicone fluids, silsesquioxanes and others.

The antimicrobial coating can be produced by reacting and/or curing an aqueous or non-aqueous composition that includes a silane or a silanol partial condensate, in combination with one or more additional ingredients, onto the surface of the article

Particularly preferred are coatings formed by employing the compounds and methods disclosed in the following U.S. Patents and Patent Applications, the teachings of which are incorporated herein by reference in their entirety:
U.S. Pat. No. 5,929,159, issued on Jul. 27, 1999, to Schutt et al;
U.S. Pat. No. 6,432,191 B2, issued on Aug. 13, 2002, to Schutt et al;
U.S. Pat. No. 6,451,382 B2, issued on Sep. 17, 2002, to Schutt et al,
U.S. Patent Application 2001/0030038 A1, published Oct. 18, 2001;
U.S. Patent Application 2001/0032568 A1, published Oct. 25, 2001;
U.S. Patent Application 2001/0056141 A1, published Dec. 27, 2001;
U.S. Patent Application 2002/0102417 A1, published Aug. 1, 2002.

Compositions and methods taught in the following U.S. Patents, the teachings of which are incorporated herein by reference in their entirety, also can be used:
U.S. Patent Application U.S. Pat. No. 3,944,702, issued on Mar. 16, 1976, to Clark;
U.S. Patent Application U.S. Pat. No. 3,976,497, issued on Aug. 24, 1976, to Clark;
U.S. Patent Application U.S. Pat. No. 3,986,997, issued on Oct. 19, 1976, to Clark;
U.S. Patent Application U.S. Pat. No. 4,027,073, issued on May 31, 1977, to Clark.

In one embodiment, living tissue or a medical article, such as, for instance a catheter, is coated using an oligomeric composition and method of coating described in U.S. Pat. No. 5,929,159, issued on Jul. 27, 1999, to Schutt, et al.

The oligomeric coating composition is aqueous-based and includes an oligomeric siloxane binder and divalent metallic (M⁺²) ions, such as, for example, Cu⁺², Zn⁺², Ca⁺², Co⁺², and Mn⁺². Upon curing, the siloxane oligomeric binder, also referred to as “siloxanol polymer” or “partial condensate of silanol” forms a silsesquioxane, e.g., methyl silane sesquioxide or CH₃SiO_{3/2}.

The siloxane oligomeric binder can be synthesized in situ, for example by hydrolysis of precursors such as, for instance, monomethylalkoxysilane, e.g., methyltrimethoxysilane (CH₃Si(OCH₃)₃) to form a partial condensate of methyl trisilanol.

The monomethylalkoxysilane also can be provided in a mixture with copolymerizable silane monomer(s).

A copolymer may be formed from cohydrolyzed silanol, RSi(OH)₃, of which methyl trisilanol comprises at least about 70% by weight, preferably at least about 75% by weight, and wherein R is a non-reactive organic moiety, such as, for example, e.g., lower alkyl, e.g., C₁-C₆ alkyl, especially C₁-C₃ alkyl, e.g., methyl, ethyl or n- or iso-propyl, vinyl, 3,3,3-trifluoropropyl, γ-glycidyloxypropy, γ-methacryloxypropyl, and phenyl.

When only methyl silanol (from methyl trialkoxysilane) is used, the amount of metal cation, (M⁺²) added can be based on the amount of silanol. When mixtures of silanol are used the molar silane sesquioxide equivalent of the remaining silane mixture can be converted to the molar equivalent of methyl silane sesquioxide.

In one example, the composition includes, on a weight basis of the total composition, from about 28% to 71%, preferably from about 31% to 71% silanol (of which at least about 70% is methylsilanol), from about 29% to about 39% water, from 0 to about 31%, preferably from about 15 to about 30%, isopropanol or other volatile organic solvent, and an M⁺² ion or a mixture of such M⁺² ions, within the range of from about 0.5 to 3 millimoles (gram x millimoles), preferably about 1.2 to 2.4 millimoles, per molar equivalent of the partial condensate calculated as methyl silane sesquioxide. The pH of the mixture is adjusted to mildly to slightly acidic, such as between 2.5 and 6.2, preferably 2.8 to 6.0, more preferably 3.0 to 6.0.

More particularly, the aqueous coating composition can include a dispersion of divalent metal cations (such as Ca⁺², Mn⁺², Cu⁺², and Zn⁺²) in a solution of water/lower aliphatic alcohol of the partial condensate of at least one silanol of the formula RSi(OH)₃ in which R is a radical selected from the group consisting of lower alkyl, vinyl, phenyl, 3,3,3-trifluoropropyl, γ-glycidyloxypropyl and γ-methacryloxypropyl, at least about 70 weight percent of the silanol being CH₃ Si(OH)₃, acid in an amount sufficient to provide a pH in the range of from about 2.5 to about 6.2, and said divalent cations in an amount of from about 1.2 millimoles to about 2.4 millimoles per molar equivalent of the partial condensate, calculated as methyl silane sesquioxide.

The coating composition can further contain colloidal silica (e.g., a 50% colloidal silica sol). Minor amounts, for instance, sufficient to provide up to about 40% of silane equivalent of the partial condensate, calculated as methyl silane sesquioxide, are preferred. Compositions that are free of colloidal silica are most preferred.

Optionally, the compositions include pigment, e.g., particulate polytetrafluoroethylene. When pigment is not included, the composition cures to a transparent glass-like finish.

The aqueous-based coating composition can be prepared by combining two components. The first component is an acidic, aqueous solution, optionally including an alcohol or other suitable volatile organic solvent. The first component includes divalent metallic ions (M⁺²), wherein M is present in an amount sufficient to provide at least about 1.2 millimoles per molar equivalent of methyl silane sesquioxide calculated from the molar silane equivalents of silane contained in the second part or component. Examples of divalent metallic cations include Ca⁺², Mn⁺², Cu⁺², Zn⁺² or mixtures thereof. Optionally, the first component can further include a hydrosol, e.g., colloidal silica, preferably in small amounts, as discussed above.

The second component is non-aqueous and contains a precursor of the siloxane oligomer. The second, non-aqueous component contains at least one silane, preferably trialkoxy silane of the formula RSi(OR¹)₃, wherein R is as defined above, and R¹ is a hydrolyzable hydrocarbyl radical, preferably a C₁-C₆ lower alkyl group, such as, methyl, ethyl, isopropyl, t-butoxy. Preferably, at least 70 percent by weight of silane is CH₃Si(OCH₃)₃. Other hydrolizable silanes also can be employed. The second component also includes a non-aqueous (organic) solvent such as isopropanol or other compatible volatile organic solvent.

The first and second components can be combined in any order. In one example, the second (silane) component is slowly added to the aqueous acidic metal cation solution, under stirring. The silane(s) in the resulting mixture hydrolyze to the corresponding silanols which condense to form a partial condensate (siloxanol polymer) and raise the pH to a final pH in the range of from about 2.5 to about 6.2, preferably from about 2.8 to about 6.0, more preferably from about 3.0 to about 6.0.

After the pH rises to the above level, the composition can be applied to a substrate, such as to a surface of a medical article. The resulting oligomeric siloxane is ready for application in a few hours and has application potential for several days. The useful application time of the mixture can be extended by chilling to a temperature exceeding the freezing point of water. After being applied, the composition dries and the siloxane oligomeric binder undergoes further condensation (curing) at ambient or elevated temperature conditions, to form a silsesquioxane, RSiO_{3/2}, where R is as defined above, predominantly methyl silane sesquioxide, CH₃SiO_{3/2}. Cure of the applied coating mixture may be accelerated by heating nominally to 80° C. Air curing at ambient conditions is preferred.

Low water levels can be employed. Thus, for example, within the general proportions set forth above, the amount of water may be maintained low to enhance the quality of the coating. Sufficient water amounts allow complete hydrolysis of the silane(s). If there is insufficient water, the siloxanes tend to cyclicize to form oily resins rather than hard films. Water levels also affect saturation of M²⁺ ions.

The volatile organic compounds (VOCs), e.g., methanol and/or isopropanol, or other lower alcohol, etc., may combine to a level no greater than 3.5 pounds per gallon in the presence of the condensate, water, ion solution and acid. However, in practice, because the coating composition is applied thinly VOC levels are, generally, not measurable.

The first component can be prepared by first determining the required aliquot of water to meet the VOC requirement and combining with the water, first the selected solution of a divalent cation followed by acidification to the desired pH (e.g., using acetic acid) after previously accounting for the possible addition of a colloidal silica sol and the neutralization of its sodium content.

The aqueous coating composition can be applied by spreading, e.g., by wiping or brushing, by spraying or by dipping. Both manual and automated methods can be used, as known in the art.

Optionally, the surface of a medical article being coated can be pre-cleaned with a suitable agent or method, as known in the art. A primer also can be employed prior to coating.

It is believed that these coating compositions utilize the reactivity of a silanol moiety with surface-oxy and/or -hydroxy species and promote the formation of a contiguous interfacial layer unaffected by surface and bulk diffusion of water, water vapor and oxygen.

The resulting medical articles have strongly adherent, transparent protective surface coatings which, depending on the porosity of the substrate medical article, may extend from about several millimeters below the surface to throughout the entirety or majority of porous substrates. It is believed that the coatings have antimicrobial properties.

A partial condensate of silanol, e.g., a partial condensate of CH₃Si(OH)₃, also can be provided in a composition that includes a dispersion of colloidal silica in a lower aliphatic alcohol-water solution, as described in U.S. Pat. Nos. 3,944,702, 3,976,497, 3,986,997 and 4,027,073.

In another embodiment, living tissue or a medical article, e.g., catheter, is produced by employing the compounds and methods disclosed in U.S. Patent Application No. 2001/0032568 by Schutt, published on Oct. 25, 2001.

Generally, the composition includes one or more silanes, e.g., methyl- or phenyl-trimethoxysilane and is formulated in combination with an acidic or basic catalyst.

In one example, the coating composition is formed by combining component (A), one or both of components (B) and (C), with component (D). Component (A) includes at least one silane, (B) is a base component, (C) is an acid component, and (D) is water.

In one embodiment, component A includes at least one silane of the formula (1):
R¹Si(OR²)₃ (1)

R¹ is alkyl, preferably, a C₁-C₆ alkyl group (the group may be a straight, cyclic, or branched-chain alkyl), such as methyl, ethyl, n- or iso-propyl, n- or iso-butyl, n-pentyl, cyclohexyl, and the like. R¹ preferably, a C₁-C₄ alkyl group, e.g., methyl, ethyl, propyl or butyl group), aryl, such as a phenyl, or a functional group. Suitable functional groups include any groups, other than hydroxyl, (including alkoxy, aryloxy, etc.), which are hydrolyzable to provide, in situ, a reactive group (e.g., reactive hydrogen) which will react, in other than a condensation reaction, with the substrate itself, or other reactive components in or generated from the coating composition.

The functional groups, in addition to the hydroxyl group (by hydrolysis of the (OR²) groups), tend to form three-dimensional or cross-linked structure, as well known in the art.

Specific examples of suitable functional groups include groups such as vinyl, acrylic, methacrylic, amino, mercapto, or vinyl chloride functional group.

Specific examples of silanes of formula (1), wherein R¹ is an alkyl group or aryl group, include, for example, methyltrimethoxysilane, ethyltrimethoxysilane, ethyltriethoxysilane, n-propyltrimethoxysilane, n-propyltriethoxysilane, isopropyltrimethoxy silane, n-butyltrimethoxy silane, isobutyltrimethoxy silane, phenyltrimethoxy silane, preferably methyltrimethoxy silane.

Specific examples of silanes of formula (1), wherein R¹ is a functional group, include, for example, of N-(2-aminoethyl)-3-aminopropyltrimethoxy silane, 3-mercaptopropyltrimethoxy silane, 3-mercaptopropyltriethoxy silane, 3-aminopropyltriethoxy silane, 3-(meth)acryloxypropyltrimethoxy silane, 3-(meth)acryloxypropyltriethoxy silane, n-phenylaminopropyltrimethoxy silane, vinyltriethyoxy silane, vinyltrimethoxy silane, allyltrimethoxy silane, and others.

Each R² is, independently, an alkyl group (i.e. a C₁-C₆ straight or branched chain alkyl group, preferably a C₁-C₄ alkyl group, such as a methyl group).

In one embodiment, component (A) includes mixtures of two or more silane compounds of formula (1). Mixtures of at least phenyltrimethoxysilane and methyltrimethoxysilane are preferred.

Generally, total amounts of silane compounds of formula (1) is within the range of from about 40 to about 90 percent by weight, preferably from about 50 to about 85 percent by weight, based on the total weight of silanes, acid component and water.

The base component (B) may be, for example, an inorganic base, such as, for example, calcium hydroxide, aluminum hydroxide or zinc hydroxide, or mixture thereof, or an organic base component, such as, for example, an aminosilane.

The amount of the base component generally is no higher than about 2%, such as, for example, from about 0.1 to 2.0%, by weight of the composition, especially, from about 0.2 to 1.6%.

In one example, component B includes a bistrifunctional silane, such as represented by the following formula 2:
X[R¹Si(OR²)₃]₂ (2)
where R¹ and R² are as defined above, and X represents an amino group (—NH) or keto group (>C═O).

Examples of aminosilane or ketosilane catalyst according to formula (2), include, bis(trimethoxypropylsilane)amine, bis(trimethoxyethylsilane)amine, di(trimethoxybutylsilane) ketone, di(trimethoxypropylsilane) ketone, and the like. The silane compounds of formula (2) function as a less active basic catalyst, and generally do not require acidic passivation. Minor amounts, usually from about 1 to about 10 parts, preferably, from about 2 to about 8 parts, of compound of formula (2) per 100 parts of silane compound(s) of formula (1) generally are expected to provide satisfactory results.

Examples of the acid component (C) include alkanoic acids, such as, for example, formic acid, acetic acid, propanoic acid, butyric acid, and inorganic acids, such as, for example, boric acid (H₃BO₃) or ortho-phosphorous acid (H₃PO₃). In one embodiment, the acid component is acetic acid, boric acid or ortho-phosphorous acid, preferably, acetic acid. The acid may be added as free acid or as an inorganic salt thereof, such as alkali metal (e.g., sodium), alkaline earth metal (e.g., calcium), or ammonium salt.

Generally, total amounts of the inorganic acid component are within the range of from about 0.3 to about 4 percent by weight, preferably from about 0.5 to about 3%, more preferably, from about 0.5 to about 2.5 percent by weight, based on the total weight of silanes, acid component and water. For acetic acid, the preferred range is from about 0.1 to about 1.0 percent, preferably, from about 0.2 to about 0.7 percent, by weight, based on the total weight of the composition.

Generally, the total amount of water is within the range of from about 10 to about 60 percent by weight, preferably from about 10 to about 45 percent by weight, based on the total weight of silanes, acid component and water.

Some or all of the water may be provided by the acid or base component, when the component is supplied as an aqueous solution, e.g., 5% aqueous solution of ortho-phosphorous acid or saturated aqueous solution of boric acid (about 6% by weight of H₃BO₃).

Since the presence of metallic and other impurities may have an adverse effect on the properties of the resulting coatings, preferably, the water is distilled or de-ionized water.

It will be recognized by those skilled in the art, that these amounts may be increased or decreased and that the optimum amounts for any particular end use application may be determined by the desired performance. In this regard, for example, when the amount of catalyst is reduced, the time to achieve freedom from tack will increase. Similarly, when the amount of the catalyst(s) is (are) increased, this may lead to increased rates of cracking, loss of adhesion and performance loss of the resulting coating.

The compositions of this embodiment may further include one or more additional additives for functional and/or esthetics effects, such as, for example, silicates, mono lower alkyl ether or ethylene glycol, epoxysilane, organic solvents and co-solvents, UV absorbers, metal catalysts and others.

The optional ingredients may be used singly or in any combination in the coating compositions of this invention.

Suitable silicates, include ethyl or methyl orthosilicate or ethyl polysilicate. These silicates may be hydrolyzed, for example, from about 28% to about 52% silica. Especially preferred in this regard is tetraethylsilicate (TEOS) which has been subjected to controlled hydrolysis, providing a mixture of TEOS and, from about 20% to about 60% polydiethoxysilane oligomers. For example, a 50% hydrolysis product may be referred to herein as “polydiethoxysilane (50%).”

Generally, total amounts of silicate component are within the range of from 0 to about 45 percent by weight, preferably from 0 to about 25 percent by weight, based on the total weight of silanes, acid component and/or base component and water.

Examples of mono-lower alkyl ether of alkylene (e.g., ethylene) glycol include mono-C₁-C₆-alkyl ethers of ethylene glycol, such as, for example, monomethyl ether, monoethyl ether, monopropyl ether, monobutylether, monopentylether or monohexylether, preferably monoethyl ether of ethylene glycol.

Generally, total amounts of the mono-lower alkyl ether of ethylene glycol are within the range of from 0 to about 15 percent by weight, preferably from 0 to about 6 percent by weight, based on the total weight of silanes, acid component and/or base component and water.

An example of ultra-violet light absorber that can be used is titanium dioxide, preferably in finely powdered form, e.g., having an average particle diameter of about 20 nanometers (nm). Other inorganic or organic ultra-violet light absorbers may be employed.

Generally, total amounts of the ultra-violet light absorber are within the range of from 0 to about 10 percent by weight, preferably from 0 to about 5 percent by weight, based on the total weight of silanes, acid component and water.

Examples of organic solvents include lower alkanol, e.g., C₂-C₄ alkanols, preferably isopropanol. Other organic solvents, such as, for example, acetone, methyl ethyl ketone, ethyl acetate, and others may also be used.

Generally, total amounts of organic solvent, such as, lower alkanol, are within a range of from 0 to about 50 percent by weight, preferably from 0 to about 30 percent by weight, based on the total weight of silane(s), acid component and/or base component and water. In some cases, higher amounts may be convenient, especially where, for example, the coating compositions are applied by spraying as an aerosol or mist.

Metal catalysts include (i) colloidal aluminum hydroxide or (ii) metal alcoholates, such as those represented by the following formula (3):
M(OR³)ₘ (3)
where M is a metal of valence m (namely, from Groups IIIA, IVA, IIB or IVB of the periodic table of the elements, e.g., boron, titanium, aluminum, indium, yttrium, cerium, lanthanum, silicon, tin, hafnium, etc; alkoxides of boron, aluminum and titanium, for instance, are readily commercially available, and tend to be non-toxic);
R³ is a lower alkyl group, e.g., C₁-C₆ straight or branched chain alkyl group, preferably C₂-C₄ alkyl group, most preferably, isopropyl, isobutyl or n-butyl; and
m is an integer of 3 or 4.

Specific examples of the metal alcoholates of formula (2), include titanium alcoholates of C₂-C₄ alkanols, e.g., titanium tetraisopropoxide and titanium tetrabutoxide.

In addition, double metal alcoholates of, for example, AlTi, AlZr, AIY, MgAl, MgTi, MgZr, etc., can also be used.

Generally, total amounts of the colloidal aluminum hydroxide and/or metal alcoholate, are within the range of from 0 to about 2.5 percent by weight, preferably from 0 to about 1 percent by weight, based on the total weight of the composition.

In one embodiment, the silane component (A) may be used in an amount of from about 15 to about 25 parts by weight, preferably as a mixture of from about 15 to about 20 parts by weight of methyltrimethoxysilane and from about 1 to about 5 parts by weight of phenyltrimethoxysilane; the base component (B), when present, is used in an amount of from about 0.1 to 3 weight percent, preferably from about 0.2 to 2.5 weight percent; the acid component (C), when present, is used in an amount of from about 0.2 to about 0.8 part by weight; the water (D) is used in an amount of from about 2.5 parts by weight to about 22 parts by weight; the silicate component is used in an amount of from 0 to about 15 parts by weight; the mono-lower alkyl ether of ethylene glycol is used in an amount of from 0 to about 3 parts by weight; the ultra-violet light absorber is used in an amount of from 0 to about 2 parts by weight; and lower alkanol is used in an amount of from 0 to about 20 parts by weight; and the colloidal aluminum hydroxide and/or the metal alcoholate is used in an amount of from 0 to about 0.5 part by weight.

In a preferred embodiment of the invention, the coating compositions include, metal catalysts which additionally provide a tint or coloration to the resulting coating. Chromium acetate hydroxide, for example, can serve as a basic catalyst which provides a bluish tint to the resulting coating. This feature may be useful for providing coatings to medical articles having large surface areas and hard to reach regions, where visibility of the applied coating can assure total coverage of the areas to be coated, while avoiding wasting coating by excessive applications over already coated surfaces.

Other basic metal catalysts providing a colorant function include, for example, iron acetate, iron acetate hydroxide, chromium acetate.

The present coating composition may be formed by mixing the above-noted components and allowing them to react. A suitable reaction time is typically 4 to 12 hours, if no colloidal aluminum hydroxide and/or metal alcoholate is present. Shorter reaction times may be obtained in the presence of colloidal aluminum hydroxide and/or metal alcoholate. If no lower alkanol is present, frequent shaking may be employed to achieve a shorter reaction time.

For ease of handling, the coating composition may be provided as a two or three container system, e.g., the silane component and any silicate component, if present, being provided in a first container and all other components being provided in a second or second and third container. The water may be provided separately from the other components. The contents of the two or three containers may be mixed shortly prior to use and allowed to react for an appropriate reaction time, as noted above.

The composition also can include one or more epoxysilane.

In one embodiment of the invention, a coating composition is prepared by combining
(A) at least one silane of the formula (1)
   R¹Si(OR²)₃ (1)
wherein R¹ and R² are as defined above,
(B) base component, especially hydroxides of calcium, zinc, and aluminum;
(E) epoxysilane; and
(D) water.

In this embodiment, the components (A), (B) and (D) are any of those described above in connection with the first embodiment. Similarly, one or more of the other optional ingredients, can be included, for example, the amino or keto silane compounds of formula (2), silicate component (F), metal alcoholate catalyst of formula (3), mono lower alkyl ether of alkylene glycol, UV absorbers, solvents and co-solvents, etc., as described above.

Suitable epoxy silanes, component (E), include, for example, glycidoxy(C₁-C₆-alkyl)(tri-C₁-C₃alkoxy)silane, such as, 3-glycidoxypropyltrimethoxysilane, 3-glycidoxypropyldiisopropylethoxysilane, (3-glycidoxypropyl)methyldiethoxysilane, 3-glycidoxypropyltriethoxysilane, and epoxy-functional silane compounds represented by the formula (4) 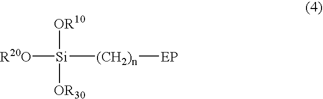
wherein R¹⁰, R²⁰ and R³⁰, independently, represent aliphatic or aromatic groups, especially, lower alkyl of from 1 to 6 carbon atoms, preferably C₁-C₃ alkyl;
EP represents glycidyl (e.g., glycidyloxy), cyclohexane oxide (epoxycyclohexyl) or cyclopentane-oxide (epoxycyclopentyl); and
n is a number of from 1 to 4, preferably 1, 2 or 3.

Examples of the epoxy functional compounds represented by formula (4), include, γ-glycidyloxymethyltrimethoxysilane, γ-glycidyloxymethyltriethoxysilane, γ-glycidoxymethyltripropoxysilane, γ-glycidoxymethyltributoxysilane, β-glycidoxyethyltrimethoxysilane, β-glycidoxyethyltriethoxysilane, β-glycidoxyethyltripropoxysilane, β-glycidoxyethyltributoxysilane, β-glycidoxyethyltrimethoxysilane, α-glycidoxyethyltriethoxysilane, α-glycidoxyethyltripropoxysilane, α-glycidoxyethyltributoxysilane, γ-glycidoxypropyltrimethoxysilane, γ-glycidoxypropyltriethoxysilane, γ-glycidoxypropyltripropoxysilane, γ-glycidoxypropyltributoxysilane, β-glycidoxypropyltrimethoxysilane, β-glycidoxypropyltriethoxysilane, β-glycidoxypropyltripropoxysilane, β-glycidoxypropyltributoxysilane, α-glycidoxypropyltrimethoxysilane, α-glycidoxypropyltriethoxysilane, α-glycidoxypropyltripropoxysilane, α-glycidoxypropyltributoxysilane, γ-glycidoxybutyltrimethoxysilane, Δ-glycidoxybutyltriethoxysilane, Δ-glycidoxybutyltripropoxysilane, Δ-glycidoxybutyltributoxysilane, Δ-glycidoxybutyltrimethoxysilane, γ-glycidoxybutyltriethoxysilane, γ-glycidoxybutyltripropoxysilane, galma-alpropoxybutyltributoxysilane, Δ-glycidoxybutyltrimethoxysilane, Δ-glycidoxybutyltriethoxysilane, Δ-glycidoxybutyltripropoxysilane, α-glycidoxybutyltrimethoxysilane, α-glycidoxybutyltriethoxysilane, α-glycidoxybutyltripropoxysilane, α-glycidoxybutyltributoxysilane, (3,4-epoxycyclohexyl)-methyltrimethoxysilane, (3,4-epoxycyclohexyl)-methyltriethoxysilane, (3,4-epoxycyclohexyl)-methyltripropoxysilane, (3,4-epoxycyclohexyl)-methyltributoxysilane, (3,4-epoxycyclohexyl)-ethyltrimethoxysilane, (3,4-epoxycyclohexyl)-ethyltriethoxysilane, (3,4-epoxycyclohexyl)-ethyltripropoxysilane, (3,4-epoxycyclohexyl)-ethyltributoxysilane, (3,4-cpoxycyclohexyl)-propyltrimethoxysilane, (3,4-epoxycyclohexyl)-propyltriethoxysilane, (3,4-epoxycyclohexyl)-propyltripropoxysilane, (3,4-epoxycyclohexyl)-propyltributoxysilane, (3,4-epoxycyclohexyl)-butyltrimethoxysilane, (3,4-epoxycyclohexy)-butyltriethoxysilane, (3,4-epoxycyclohexyl)-butyltripropoxysilane, (3,4-epoxycyclohexyl)-butyltributoxysilane.

The amount of the components (A), (B) and (D) may generally be the same amounts as previously disclosed.

The amount of component (E) epoxysilane will generally be within the range of from about 1 to about 22 percent by weight, preferably, from about 2 to 16% by weight, based on the total weight of the composition.

In still another embodiment of the invention, an aqueous silane-based coating composition is formed by combining as component (A) at least one organosilane of formula (1), as given above; (D) water; (J) (i) colloidal aluminum hydroxide, (ii) metal alcoholate of the previously given formula (3) or a mixture of (i) and (ii). Additional silane hydrolyzing catalyst, including, for example, a compound of formula (2), or any of the other disclosed aminosilane or ketosilane catalysts, effective to inhibit gellation, may also be added in order to inhibit gellation and, thereby extend storage life and pot life.

Examples of the organosilanes of formula (1) and metal alcoholate of formula (3) are as mentioned above. Generally, the amount of organosilane(s) of formula (1) will be from about 10% to 50%, preferably, 12% to 35%, and the amount of the component (J) will be from about 0.05 to about 1.0 percent, preferably, from about 0.1 to about 0.8 percent, each based on the total weight of the composition

Additional silane hydrolyzing agent and gellation inhibitors preferably include the aforementioned epoxide silanes (E).

In another embodiment, an aqueous organosilane coating composition is formed by combining (A) at least one organosilane of formula (1); (H) lower alkanol solvent; (D) water; and (K) chromium acetate hydroxide or other silane polymerization catalyst which will provide coloration to the resulting coating.

The amount of the component (K) is, usually, up to about 2 percent by weight of the coating composition, preferably from about 0.1 to about 1.8%, especially, from about 0.4 to about 1.3% by weight, based on the total weight of the coating composition.

One or more optional ingredients, such as those discussed above may also be included in the compositions of this embodiment.

In a further embodiment of the invention, an aqueous based organosilane coating composition is formed by admixing (A) at least one organosilane of formula (1); (D) water; (F) alkali metal silicate, preferably pre-hydrolyzed; (H) lower alkanol solvent; (J) the aforementioned metal catalyst (i) colloidal aluminum hydroxide, (ii) metal alcoholate of formula (3) as given above, or (iii) mixture of (i) and (ii).

The components of the compositions of this embodiment, like those of the previous alternatives, may be selected from the same components and in the same amounts as previously described.

In yet another embodiment of the invention, an aqueous organosilane coating composition is formed by combining (A) at least one organosilane of formula (1); (B′) at least one basic amine silane catalyst, (D) water, (E) epoxide silane: and (H) lower alkanol solvent.

Examples of the basic amine silane catalyst, (B′), include, but are not limited to, aminoethyltriethoxysilane, β-aminoethyltrimethoxysilane, β-aminoethyltriethoxysilane, β-aminoethyltributoxysilane, β-aminoethyltripropoxysilane, α-aminoethyltrimethoxysilane, α-aminoethyltriethoxysilane, γ-aminopropyltrimethoxysilane, γ-aminopropyltriethoxysilane, γ-aminopropyltributoxysilane, γ-aminopropyltripropoxysilane, β-aminopropyltrimethoxysilane, β-aminopropyltriethoxysilane, α-aminopropyltripropoxysilane, α-aminopropyltributoxysilane, α-aminopropyltrimethoxysilane, α-aminopropyltriethoxysilane, α-aminopropyltributoxysilane, α-aminopropyltripropoxysilane, N-aminomethylaminoethyltrimethoxysilane, N-aminomethylaminomethyltripropoxysilane, N-aminomethyl-β-aminoethyltrimethoxysilane, N-aminomethyl-β-aminoethyltriethoxysilane, N-aminomethyl-β-aminoethyltripropoxysilane, N-aminomethyl-γ-aminopropyltrimethoxysilane, N-aminomethyl-γ-aminopropyltriethoxysilane, N-aminomethyl-γ-aminopropyltripropoxysilane, N-aminomethyl-β-aminopropyltrimethoxysilane, N-aminomethyl-β-aminopropyltriethoxysilane, N-aminomethyl-β-aminopropyltripropoxysilane, N-aminopropyltripropoxysilane, N-aminopropyltrimethoxysilane, N-(β-aminoethyl)-β-aminoethyltrimethoxysilane, N-(β-aminoethyl)-β-aminoethyltriethoxysilane, N-(β-aminoethyl)-β-aminoethyltripropoxysilane, N-(β-aminoethyl)-β-aminoethyltrimethoxysilane, N-(β-aminoethyl)-α-aminoethyltriethoxysilane, N-(β-aminoethyl)-α-aminoethyltripropoxysilane, N-(β-aminoethyl)-β-aminopropyltrimethoxysilane, N-(β-aminoethyl)-γ-aminopropyltriethoxysilane, N-(β-aminoethyl)-γ-aminopropyltripropoxysilane, N-(β-aminoethyl)-γ-aminopropyltrimethoxysilane, N-(β-aminoethyl)-β-aminopropyltriethoxysilane, N-(β-aminoethyl)-β-aminopropyltripropoxysilane, N-(γ-aminopropyl)-β-aminoethyltrimethoxysilane, N-(γ-aminopropyl)-β-aminoethyltriethoxysilane, N-(γ-aminopropyl)-β-aminoethyltripropoxysilane, N-methyl aminopropyltrimethoxysilane, β-aminopropylmethyldiethoxysilane, γ-diethylene triaminepropyltriethoxysilane, and the like.

Of these, 3-(2-aminoethylamino)propyltrimethoxy silane (also known as N-(2-aminoethyl)-3-aminopropyltrimethoxysilane), and 3-aminopropyltrimethoxy silane, are preferred.

Aminosilanes of formula (2), above, can also be used.

For the components (A), (D), (E) and (H), the representative examples and amounts given above can also be used in this embodiment.

In another embodiment of the invention, a multivalent catalyst system may be used to polymerize the organosilane of formula (1). For example, the mixed multivalent catalyst may include a divalent metal compound, such as hydroxide or carbonate of calcium, magnesium or other alkaline earth metal; a trivalent metal compound, such as, for example, boric acid or other compound of boron or aluminum; and a tetravalent metal compound, such as a compound of formula (3-a):
M¹-(OR³)₄ (3-a)
where M¹ represent a tetravalent metal, such as titanium, or zirconium, and R³ is as previously defined.

According to this embodiment, the proportions of the respective catalysts may be selected based on the desired properties but generally in terms of metal ions, weight ratios of M⁺²:M⁺³:M⁺⁴ of from about 0.1-1:0.05-1:0.1-2, preferably from about 0.4-1:0.2-1:0.5-1, are expected to provide good results. In one example of the invention, the coating composition contains as the major film-forming components, a mixture of one or more silane compounds of above formula (1), wherein R¹ in a first silane compound is a lower alkyl group, such as methyl or ethyl while in a second silane compound, R¹ is an aryl group, especially, phenyl. Additional silanes can be included. In one embodiment, the ratio of the first silane compound to second silane compound is within a range of from about 3:1 to about 1:3, preferably, from about 1.5:1 to about 1:1.5, such as about 1:1, on a weight basis.

This composition also can include a small amount of moderately alcohol soluble to alcohol soluble basic activator for the silanes (either in the container, or in situ), especially, calcium hydroxide or tetramethylammonium hydroxide. Generally, the amount of calcium hydroxide, is in the range of from about 0.4 to about 4, preferably, from about 1.2 to about 2.8 parts of basic activator, per 100 parts, in total, of silane compounds of formula (1). Since tetramethylammonium hydroxide tends to be more active and more soluble in alcohol than calcium hydroxide, smaller amounts of this basic activator, can be used, for example, from about 0.01 to about 2, preferably, from about 0.02 to about 1 part of tetramethylammonium hydroxide, per about 100 parts of silane compounds of formula (1).

The formulation also includes a silicate, preferably, partially hydrolyzed silicate, such as, for example, hydrolysis product of tetraethylsilicate, e.g., polydiethoxysiloxane (about 50% solids). Amounts of the silicate, on a solids basis, per 100 parts of silane compounds of formula (1), generally are within the range of from about 1 to 16 parts, preferably, from about 2 to about 10 parts, more preferably, from about 4 to about 8 parts.

The film-forming and catalyst ingredients are added to lower alcohol solvent, preferably, isopropyl alcohol. Relatively dilute solutions facilitate application by wiping (e.g., using a soft cloth, sponge, etc.) or spraying. Generally, from about 600 up to about 1500 parts of alcohol per 100 parts of silane compounds of formula (1) provide satisfactory results.

An optional ingredient for this formulation is γ-glycidyloxypropyltrimethoxysilane, or other epoxy silane compound, such as mentioned above.

In some cases, monovalent alkalis, such as, for example, sodium hydroxide, potassium hydroxide, and the like can be too active for easy application of the composition, while other less active alkalies, require addition of acid catalyst to promote the reaction.

It is believed that the coating compositions of this invention, when applied to a substrate, such as a medical article, will readily penetrate even narrow and microscopic crevices or pores of the substrate, to form strong adherent bonds with the substrate. Although not wishing to be bound by any particular theory of operation, it is believed that the penetration and adherent bond formation is achieved, in part, because of the absence of large organic molecules from the coating compositions. It is further believed that the resulting coatings have antimicrobial properties.

The coating compositions described herein can be formulated as solventless, aqueous or non-aqueous systems (although, in most cases, at least a catalytic amount of water is eventually added, directly or taken from the atmosphere). For example, the solventless systems may contain a mixture of methyltrimethoxysilane and phenyltrimethoxysilane and catalyst, e.g., metal alcoholate, such as, for instance, tetrabutoxytitanate. Suitable non-aqueous systems (e.g., by addition of small amounts of diluent, especially, lower alcohol, such as, isopropanol), may also be used.

The coating composition may be applied in any conventional manner, for instance by dipping, wiping, brushing or spraying. Preferably, the spraying is carried out under an inert atmosphere, especially using dry N₂ propellant, as a result of which extra gloss and hardness is imparted to the resulting coating. Although the reason for this has not been ascertained, it is believed that nitrogen gas impacting the substrate surface removes at least some of the adsorbed oxygen and water, while at the same time, its positive Joule-Thomson coefficient retards solvent evaporation and promotes film generation. Therefore, since any adsorbed oxygen and/or water would be expected to impair the qualities of the resultant coating, removal of these species by the N₂ gas stream, would tend to improve the qualities of the coating, including gloss and hardness.

The present invention also provides coating compositions that can serve as primer compositions.

A primer composition can be prepared by combining two or more polyfunctional organosilanes as previously described. Monofunctional (e.g., organosilanes of formula (1) where R¹ is alkyl or aryl) are not included in the primer composition. Silica and silicate or precursors thereof are also not included in the primer compositions.

At least one of the polyfunctional organosilanes will preferably include polyamino group as R¹ in formula (1), namely, aminosilanes of the following formula (1-A)
H₂N—Rₐ—NH—R_{b}—Si(OR²)₃ (1-A)
where Rₐ and R_{b} are each, independently, alkyl of from 1 to 6 carbon atoms, preferably 1 to 4 carbon atoms, especially 2 to 4 carbon atoms; and R² is as defined above.

Aminoethylaminopropyltrimethoxysilane, aminoethylaminobutyltrimethoxysilane, aminoethylaminopropyltriethoxysilane, are examples of aminosilane compounds of formula (1-A).

Other polyfunctional organosilanes, such as vinylsilanes, acrylic silanes, methacrylic silanes, and the like, as described above, may be used in place of, or in addition to, the polyaminopolyfunctional organosilanes.

At least one other polyfunctional organosilane which is an epoxysilane, including any of the epoxysilanes mentioned above, also can be included as one of the polyfunctional organosilanes of the primer coating composition. Preferred are the glycidyloxy(C₁-C₆-alkyl)(tri-C₁-C₃ alkoxy) silanes, such as 3-glycidyloxypropyltrimethoxysilane.

Suitable amounts of the polyfunctional organosilanes in the primer coating composition, as solids (non-volatile silanol condensation products after addition of water) will generally range from about 1% to about 20%, by weight, preferably from about 2% to about 15%, more preferably from about 4% to 12%, by weight, of the composition. These amounts, for a primer containing two of the polyfunctional silane compounds, generally correspond to from about 2% to about 40%, preferably from about 4% to about 30%, more preferably from about 8% to about 24%, by weight, of the total composition, before water addition.

In one composition containing a mixture of (a) polyaminoorganosilane and (b) glycidyloxyorganosilane, in appropriate volatile organic solvent, preferably isopropyl alcohol, the total amount of (a) plus (b) is preferably from about 5 to about 25 parts, more preferably, from about 10 to about 20 parts, per 100 parts of volatile organic solvent. Furthermore, weight ratios of (a):(b) in the range of from about 1:0.4 to 2, preferably 1:0.6 to 1.4, are expected to provide good results.

In use, the organic solvent, e.g., isopropyl alcohol, solution of the polyfunctional organosilanes, is thoroughly mixed with a small amount of water to catalyze the hydrolysis reaction. The resulting formulation is then generally ready to be applied to the substrate medical article in from about 5 minutes to about 1 hour, typically, in about 10 to about 30 minutes, such as about 15 minutes.

A minor amount of water, such as from about 1 to 2 parts water per 100 parts of solution (e.g., from about 0.5 to 1.0 parts water per 10 parts polyfunctional silanes) will be sufficient to catalyze the hydrolysis reaction.

The primer composition may be applied to the substrate in any convenient manner, such as by wiping, brushing, dipping or spraying. Since the subject primer coating compositions do not form lumps, it is easy and convenient to apply by spraying, for example, using a number 4-6 nozzle with a pressure of about 20 psi. Preferably, the spray is in the form of a mist. It is not necessary to apply the composition uniformly since the coating will tend to flow together to provide a uniform continuous film.

Although the precise nature of the resulting primer coating is not known, it is believed that the polyfunctional silanes provide multiple reactive sites for adhering to the substrate, to itself and each other, as well as to the subsequently applied top coat. Presumably, because of the strong adhesion to the substrate, resulting from the multiple reactive sites, the primer coating is resistant to chemical attack. Accordingly, the primer coating compositions of this invention can be used in applications where exposure to alkali and/or acidic agents is anticipated.

Thin coatings, on the order of about 2000 nm, or less, are expected to provide good results. Generally, the coating compositions of the present invention are effective when applied to a coating (film) thickness (after cure) in the range of from about 5 to about 150 millionths of an inch, however, if desired, thicker films may be applied.

In another embodiment the coating on living tissue or on the surface of a medical article is formed using the compounds and methods taught in U.S. Patent Application No. 2001/0056141, by Schutt, published on Dec. 27, 2001.

The compositions disclosed therein are non-aqueous coating compositions of oligomeric siloxane binder and a catalyst which promotes hydrolysis and which can become an integral part of the siloxane network.

The coating is formed by combining
(A) at least one silane of formula (1)
   R¹ₙSi(OR²)₄₋ₙ (1)
wherein R¹ represents a lower alkyl group, a phenyl group or a functional group containing at least one of vinyl, acrylic, amino, mercapto, or vinyl chloride functional groups;
R² represents a lower alkyl group; and,
n is a number of 1 to 2; and
(B) at least one compound selected from the group consisting of
   (i) vinyltriacetoxysilane and/or
   (ii) colloidal aluminum hydroxide and/or
   (iii) at least one metal alcoholate of formula (3)
      M(OR³)ₘ (3)
wherein M represents a metal of valence m,
R³ represents a lower alkyl group, and
m is a number of 2 to 4.

In one example, the non-aqueous coating composition is formed by combining components (A) and (B), as set forth above, and components (C) at least one silica component selected from the group consisting of methyl orthosilicate, ethyl orthosilicate, ethylpolysilicate and colloidal silica dispersed in lower alcohol and (D) an acid component selected from the group consisting of boric acid and boric acid dissolved in lower alcohol.

The non-aqueous coating composition also can be formed by combining components (A), (B) and (D), as set forth above, with the proviso that a mixture of silane compounds of formula (1) is used, wherein at least one silane compound that has R¹γ-glycidyloxypropyltrimethoxy is present in the mixture.

In the silanes of formula (1) R¹ is alkyl, preferably, a C₁-C₆ alkyl group (the group may be a straight, cyclic, or branched-chain alkyl), such as methyl, ethyl, n- or iso-propyl, n- or iso-butyl, n-pentyl, cyclohexyl, and the like, preferably a q-C₄ alkyl group, most preferably a methyl, ethyl, propyl or butyl group), aryl, such as a phenyl, or a functional group or groups, such as vinyl, acrylic, methacrylic, amino, mercapto, or vinyl chloride functional group, e.g., 3,3,3-trifluoropropyl, γ-glycidyloxypropyl, γ-methacryloxypropyl, N-(2-aminoethyl)-3-aminopropyl, aminopropyl, and the like; and each R² is, independently, an alkyl group (i.e. a C₁-C₆ straight or branched chain alkyl group, preferably a C₁-C₄ alkyl group, such as a methyl group).

Examples of silanes of formula (1), wherein R¹ is an alkyl group or aryl group, and n is 1, include methyltrimethoxysilane, ethyltrimethoxysilane, ethyltriethoxysilane, n-propyltrimethoxysilane, n-propyltriethoxysilane, isopropyltrimethoxysilane, n-butyltrimethoxysilane, isobutyltrimethoxysilane, phenyltrimethoxysilane, preferably methyltrimethoxysilane, phenyltrimethoxysilane, and mixtures thereof. In the case where R¹ is a functional group, mention may be made, for example, of N-(2-aminoethyl)-3-aminopropyltrimethoxysilane, 3-mercaptopropyltrimethoxysilane, 3-mercaptopropyltriethoxysilane, 3-aminopropyltriethoxysilane, 3-(meth)acryloxypropyltrimethoxysilane, 3-(meth)acryloxypropyltriethoxysilane, n-phenylaminopropyltrimethoxysilane, vinyltriethyoxysilane, vinyltrimethoxysilane, allyltrimethoxysilane, γ-glycidyloxypropyltrimethoxysilane, and others, e.g., any of the aminosilane catalysts, described herein.

When n is 2, the silane compounds may be represented by, for example, dimethyldimethoxysilane, diethyldimethoxysilane, diphenyldimethoxysilane, methylethyldimethoxysilane, divinyldimethoxysilane, methyl-γ-glycidyloxypropyldimethoxysilane, and the like.

Examples of functional groups include any group, other than hydroxyl, (including alkoxy, aryloxy, etc.), which is hydrolyzable to provide, in situ, a reactive group (e.g., reactive hydrogen) which will react, in other than a condensation reaction, with the substrate itself, or other reactive components in or from the coating composition. The functional groups, in addition to the hydroxyl group (by hydrolysis of the (OR²) groups), tend to form three-dimensional or cross-linked structure, as known in the art.

Mixtures of two or more silane compounds of formula (1) can be used. Mixtures of at least phenyltrimethoxysilane and methyltrimethoxysilane are preferred.

Generally, total amounts of silane compounds of formula (1) are within the range of from about 50 to about 99.6 percent by weight, preferably from about 60 to about 98 percent by weight, more preferably, from about 70 to about 97.5%, by weight, based on the total weight of the composition.

The component (B) functions as a catalyst for the silane component (A). For metal alcoholates (B)(ii) is represented by the following formula (3):
M(OR³)ₘ (3)
where M is a metal of valence m (namely, from Groups IIIA, IVA, IIB or IVB of the periodic table of the elements), e.g., boron, titanium, aluminum, indium, yttrium, cerium, lanthanum, silicon, tin, hafnium, etc; boron, aluminum and titanium, for example, are commercially available, and tend to be non-toxic.

R³ is a lower alkyl group, e.g., C₁-C₆ straight or branched chain alkyl group, preferably C₂-C₄ alkyl group, most preferably, isopropyl, isobutyl or n-butyl.

Specific examples of the metal alcoholates of formula (3), include metal alcoholates of C₂-C₄ alkanols, e.g., titanium tetraisopropoxide (also may be referred to as tetraisopropoxytitanate), titanium tetrabutoxide, aluminum triisopropoxide, zinc diisopropoxide, zinc di-n-butoxide, calcium diisopropoxide, calcium diisobutoxide, boron triisopropoxide, boron triisobutoxide, and the like.

In addition, double metal alcoholates of, for example, AlTi, AlZr, AlY, MgAl, MgTi, MgZr, etc., may also be used.

Mixtures of two or more metal alcoholates, and mixtures of metal alcoholate(s) with vinyltriacetoxysilane and/or colloidal aluminum hydroxide, or mixture of vinyltriacetoxysilane with colloidal aluminum hydroxide, may also be used as component (B).

Generally, total amounts of component (B) will be in the range of from about 0.4% to about 10% by weight, preferably, from about 0.6% to about 4%, by weight, based on the total weight of the composition.

Depending on the particular application one or more additional components can be added to the compositions of this invention, for example, components (C) and (D).

Component (C) is a silica component which may be methylorthosilicate, ethylorthosilicate, polyethylsilicate or colloidal silica. These silicates may be hydrolyzed, for example, from about 28% to about 52% silica. Especially preferred in this regard is tetraethylsilicate (TEOS) which has been subjected to controlled hydrolysis, providing a mixture of TEOS and, from about 20% to about 60% polydiethoxysilane oligomers. For example, a 50% hydrolysis product may be referred to herein as “polydiethoxysilane (50%).” When colloidal silica is used, it will be present in an appropriate solvent medium, preferably a lower alkanol, such an isopropanol.

Generally, total amounts of silicate component (C), is within a range of from 0 to about 50 percent by weight, preferably from 0.4% to about 45% by weight, more preferably, from about 2 to about 44 wt. %, based on the total composition.

Component (D) is an inorganic acid, especially boric acid, H₃BO₃, (which may be dissolved in a solvent, such as lower (C, to C₆, preferably C₁ to C₄, alcohol, e.g., isopropanol). Phosphorous acid, H₃PO₃, also may also be used, in place of some or all of the boric acid. Aliphatic acids, such as lower alkanoic acids, e.g., formic acid, acetic acid, propanoic acid, butyric acid, and others. Acetic acid is preferred.

Boric acid component (D), when present, generally is within a range of from about 5 to about 50 wt. %, preferably, from about 8 to about 40 wt. %, based on the total weight of the composition.

In one example, the coating composition of this invention preferably include component (A) silane of formula (1), which will include γ-glycidyloxypropyltrimethoxysilane and at least one other silane of formula (1), especially methyltrimethoxysilane or mixture of methyltrimethoxysilane and phenyltrimethoxysilane. The component (D), boric acid (or solution thereof in lower alkanol, also can be included. Component (C) silicate may also be present in the composition. Suitable amounts of γ-glycidyloxypropyltrimethoxysilane generally are within a range of from about 2 to about 25 wt. %, preferably from about 5 to 20 wt. %, based on the total composition. Usually, the total amount of silane compounds of formula (1) to form a silicate overcoating generally are in the ranges specified above for the silane of formula (1).

In another example non-aqueous coating composition can be formed by combining components (A) and (B), as set forth above, and (E) finely divided solid lubricant.

Examples of finely divided solid lubricants, include graphite, molybdenum disulfide, polytetrafluoroethylene, and others. Mixtures of these solid lubricants are also useful.

When present, the amount of component (E), solid lubricant, generally is in a range of from about 5 to about 40 wt. %, preferably from about 7 to 30 wt. %, especially, from about 10 to about 28 wt. %, based on the total composition. Within these ranges, it is expected that the desired degree of resistance to adhesion of, for example, micro-organisms, will be obtained, without impairing other desired properties or curability of the composition.

A non-aqueous coating also can be formed by using a mixture of silane compounds of formula (1) wherein R¹ in one silane compound is lower alkyl and in another silane compound R¹ is aryl, especially phenyl together with a small amount of (F) calcium hydroxide and a silicate component (C), preferably, partially hydrolyzed silicate, especially a hydrolysis product of tetraethylsilicate.

Component (F) calcium hydroxide and component (C) silicate, can be added to a mixture of tri- or di-alkyloxysilanes and tri-or di-aryloxysilanes, preferably, trialkoxysilane and triaryloxysilane, according to formula (1). In this case, the amount of (F) calcium hydroxide, can be in a range of from about 0.1 to about 5 parts by weight, preferably, from about 0.5 to about 3 parts by weight, especially, from about 0.8 to about 2.5 parts by weight, based on the total weight of components (A), (B), (C), (D), (E) and (F).

The compositions can be prepared by combining the ingredients in a single container by simple mixing. When applied to a substrate, the mixture hydrolyzes thereon and chemically attaches to the substrate while simultaneously forming a strongly adherent film coating. Because the mixture of film formers is water-free when applied, mixing creates a one container system and shelf life generally does not present a problem. Attaining a tack-free state, followed by cure, can occur in about two hours for most formulations. However, since the components react with ambient moisture, care may be taken to avoid contact with such moisture prior to actual mixing and use. Any conventional technique for moisture avoidance may be utilized, e.g., vacuum packaging, hermetic seals, dry atmospheres, etc.

The nonaqueous coating compositions described above, when applied to a medical article will form a hard, abrasion resistant, flexible, and generally transparent, and corrosion resistant surface coating. The composition may be applied by any suitable technique, e.g., spraying, dipping, brushing, wiping, and the like, using automatic or manual applicators. Proper surface preparation can be performed prior to applying the coating composition.

Most embodiments of the invention become tack-free in less than two hours. Curing can be accelerated by applying heat to a level of, for example, about 80° C.

The resulting coated articles have strongly adherent, non-porous transparent protective surface coatings, which, depending on the porosity of the substrate, may extend from just below the surface for smooth surface materials, to throughout the entirety or majority of porous substrates. It is believed that the coatings have antimicrobial properties.

Although the non-aqueous compositions of the present invention are often formulated without addition of solvent, or with solvent added only as a component of another ingredient, e.g., (C) silica dispersion in lower alcohol, (D) boric acid solution in lower alkanol, etc, it is also within the scope to formulate the subject compositions as solvent-based compositions, by separately adding (G) solvent. When present in the compositions of this invention, whether added separately, or as part of another ingredient, total amounts of solvents usually are within a range of from about 0 to 1000 parts, preferably from about 0 to about 800 parts by weight, based on the total weight of the composition. In particular, solvent (G) will be included in the case of the formulations for providing hard, clear and glossy corrosion resistant coatings, to facilitate the application of the coating by wiping with a sponge or cloth.

Examples of organic solvents include lower alkanol, e.g., C₂-C₄ alkanols, preferably isopropanol. Other organic solvents, such as, for example, acetone, methyl ethyl ketone, ethyl acetate, and the like may also be used.

Generally, total amounts of organic solvent, such as, lower alkanol, are within a range of from 0% to about 50% by weight, preferably from 0% to about 30% by weight, based on the total weight of components (A)-(F). In some cases, however, such as the glossy coating compositions, substantially higher amounts can be used, especially where, for example, the coating compositions are applied by spraying as an aerosol or mist or when a lower viscosity is desirable.

Within the above mentioned amounts and proportions, and when used in any of the various embodiments, preferred amounts (parts by weight) of the respective ingredients generally are within the following ranges (based on a total of 100 parts by weight of the composition): silane component (A) from about 15 to about 25 parts of methyltrimethoxysilane, from about 1 to about 5 parts of phenyltrimethoxysilane, from about 0.3 to about 3 parts γ-glycidyloxypropyltrimethoxysilane; catalyst component (B) from about 0.2 to about 0.5 parts; silicate component (C) from about 0.2 to about 1 part; boric acid component (D) from about 0.1 to about 1 part, as H₃BO₃; solid lubricant (E) from about 2.5 to 20 parts by weight.

It will be recognized by those skilled in the art, that these amounts may be increased or decreased and that the optimum amounts for any particular end use application may be determined by the desired performance. In this regard, for example, when the amount of catalyst is reduced, the time to achieve freedom from tack will increase. Similarly, when the amount of the catalyst(s) is (are) increased, this may lead to increased rates of cracking, loss of adhesion and performance loss of the resulting coating.

The compositions of this embodiment may further include one or more additional additives for functional and/or esthetics effects, such as, for example, UV absorbers, co-solvents, such as, for example, mono-lower alkyl ether of alkylene (e.g., ethylene) glycol, and others.

As examples of mono-lower alkyl ether of alkylene (e.g., ethylene) glycol, mention may be made of mono-C₁-C₆-alkyl ethers of ethylene glycol, such as, for example, monomethyl ether, monoethyl ether, monopropyl ether, monobutylether, monopentylether or monohexylether, preferably monoethyl ether of ethylene glycol.

Example of ultra-violet light absorbers include, for example, titanium dioxide in finely powdered form, e.g., having an average particle diameter of about 20 nanometer (nm). Other inorganic or organic ultra-violet light absorbers may be employed.

Generally, total amounts of the ultra-violet light absorber, when used, generally are within the range of from 0% to about 10% by weight, preferably from 0% to about 5% by weight, based on the total weight of components (A)-(F).

Generally, total amounts of the mono-lower alkyl ether of ethylene glycol, when used, are within the range of from 0% to about 15% by weight, preferably from 0% to about 6% by weight, based on the total weight of components (A)-(F).

The medical article also can be formed using compounds and methods described in U.S. Application No. 2001/0030038 A1, by Schutt, et al., published on Oct. 18, 2001.

In the preferred embodiment of the invention, the coating composition is an aqueous or non-aqueous oligomeric silane coating composition formed by combining or mixing
(a) at least one silane of the formula (1)
   R¹ₙSi(OR²)₄₋ₙ (1)
   where R¹ represents a lower alkyl group, a C₆-C₈ aryl or a functional group including at least one of vinyl, acrylic, amino, mercapto, or vinyl chloride functional groups;
(b) silane condensation catalyst, and
(c) lower alkanol solvent, and optionally, one or more of
(d) colloidal aluminum hydroxide;
(e) metal alcoholate of formula (2):
   M(OR³)ₘ (2)
where M is a metal of valence 2, 3 or 4, or mixture of two or more such metals;
R represents a lower alkyl group; and,
m represents a number of 2, 3 or 4;
(f) a silica component selected from the group consisting of alkali metal silicate, ethyl orthosilicate, ethyl polysilicate, and colloidal silica dispersed in lower alkanol;
(g) color forming silanol condensation catalyst;
(h) epoxysilane; and,
(i) ultrafine titanium dioxide ultraviolet light absorber.

Water and co-solvent also can be included.

Examples of the above-listed components and suitable amounts are as described above.

Preferably, the above described aqueous or non-aqueous oligomeric silane coating composition containing the silane of formula (1), silane condensation catalyst and solvent, and one or more optional ingredients, is applied to the article surface. The coating composition is allowed to cure for example to a film thickness of from about 5 to about 150 millionths of an inch, while also filling microvacancies that may be present on the surface.

The coating compositions generally can be characterized as low molecular weight oligomeric silane based coatings. As used in this context the term “silane” is intended to include not only silanes but also silanols and siloxanes and the low molecular weight partial condensation products thereof. The term “low molecular weight” is intended to refer to the general absence of large or bulky organic molecules or moieties as part of the silane components, namely, wherein the organic substituents are generally limited to lower alkyl groups, especially alkyl groups containing from 1 to 4 carbon atoms, especially, 1 to 3 carbon atoms, including, in particular, methyl, ethyl, n-propyl and iso-propyl groups, and aryl groups of no more than about 8 carbon atoms, especially, no more than about 6 carbon atoms, such as, for example, phenyl, benzyl, and phenylethyl.

Still further, the coating compositions of this invention are characterized by low viscosity to facilitate the penetration into the microcrevices and microvoids that can be present on the surface of the medical article. As used herein, “low viscosity” refers to the ability to penetrate into micron and submicron size voids. Typically, the coating compositions are characterized by a coating viscosity, measured using a No. 2 (#2) Zahn Cup, of from about 4 to about 10 seconds, especially, from about 5 to about 8 seconds, measured at room temperature (approximately 18° C.).

It is believed that the coating compositions used in the present invention are capable of filling small nanometer size voids under driving forces of capillary action and Helmoltz free energy, displacing gasses and/or reacting with water or other chemicals. The ability of the coating compositions of this invention to migrate and penetrate capillary structures releasing Helmoltz free energy allows them to displace molecules bonded by means of secondary and tertiary valence forces and provide protection by forming micron range thickness coatings, on the order of from about 5 to about 150 millionths of an inch.

Specific examples of suitable oligomeric silane coating compositions useful in the present invention are described briefly below.

I. An aqueous coating composition comprising a dispersion of divalent metal cations in lower aliphatic alcohol-water solution of the partial condensate of at least one silanol of the formula RSi(OH)₃, wherein R is a radical selected from the group consisting of lower alkyl, or C₆-C₈ aryl or a functional group including at least one of vinyl, acrylic, amino, mercapto, or vinyl chloride functional groups (e.g., 3,3,3-trifluoropropyl, γ-glycidyloxypropyl, and γ-methacryloxypropyl), at least about 70 percent by weight of the silanol being CH₃Si(OH)₃, acid in amount to provide a pH in the range of from about 2.5 to about 6.2, said divalent metal cations being present in an amount of from about 1.2 millimoles to about 2.4 millimoles, per molar equivalent of the partial condensate, calculated as methyl silane sesquioxide.

II. An aqueous coating composition formed by combining or mixing
(A) at least one silane of the formula (1)
   R¹Si(OR²)₃ (1)
wherein
R¹ is a lower alkyl group, a C_{6-C8} aryl group or an N-(2-aminoethyl)-3-aminopropyl group, and
R² is a lower alkyl group;
(B) an acid component selected from the group consisting of water-soluble organic acids, H₃BO₃ and H₃PO₃; and

(D) water.

III. A non-aqueous coating composition formed by mixing
(A) at least one silane of formula (1)
   R¹ₙSi(OR²)₄₋ₙ (1)
wherein R¹ represents lower alkyl, C_{6-C8} aryl, 3,3,3-trifluoropropyl, γ-glycidyloxypropyl, γ-(meth)acryloxyp-ropyl, N-(2-aminoethyl)-3-aminopropyl, or aminopropyl group;
R³ represents lower alkyl group; and
n is a number of 1 to 2; and
(E) (i) vinyltriacetoxysilane, (ii) colloidal aluminum hydroxide; and/or (iii) at least one metal alcoholate of formula (3)
   M(OR³)ₘ (3)
wherein M represents a metal of valence m,
R³ represents lower alkyl group; and
m is a number of 2, 3 or 4.

IV. A non-aqueous coating composition formed by combining
(A) at least one silane of formula (1)
   R¹ₙSi(OR²)₄₋ₙ (1)
wherein R¹ represents lower alkyl, C_{6-C8} aryl, 3,3,3-trifluoropropyl, γ-glycidyloxypropyl, γ-(meth)acryloxypropyl, N-(2-aminoethyl)-3-aminopropyl, or aminopropyl group;
R² represents lower alkyl or acetyl group; and
n is a number of 1 to 2;
(B) boric acid, optionally dissolved in lower alkanol;
(E) (i) vinyltriacetoxysilane, (ii) colloidal aluminum hydroxide; and/or (iii) at least one metal alcoholate of formula (3)
   M(OR³)ₘ (3)
wherein M represents a metal of valence m,
R³ represents lower alkyl group
m is an number of 2, 3 or 4; and,
(F) silica component selected from the group consisting of ethyl ortho-silicate, ethyl polysilicate and colloidal silica, dispersed in lower alkanol.

V. A non-aqueous coating composition formed by combining
(A) at least one silane of formula (1)
   R¹ₙSi(OR²)₄₋ₙ (1)
wherein R¹ represents lower alkyl, C_{6-C8} aryl, 3,3,3-trifluoropropyl, γ-(meth)acryloxypropyl, N-(2-aminoethyl)-3-aminopropyl, or aminopropyl group;
R² represents lower alkyl or acetyl group; and
n is a number of 1 to 2;
(A′) γ-glycidyloxypropyltrimethoxysilane;
(B) boric acid, optionally dissolved in lower alkanol;
(E) (i) vinyltriacetoxysilane, (ii) colloidal aluminum hydroxide; and/or (iii) at least one metal alcoholate of formula (3)
   M(OR³)ₘ (3)
wherein M represents a metal of valence m,
R³ represents lower alkyl group
m is an number of 2, 3 or 4.

VI. An aqueous coating composition formed by mixing
(A) at least one silane of formula (1)
   R¹ₙSi(OR²)₄₋ₙ (1)
wherein R¹ represents lower alkyl, C_{6-C8} aryl, or a functional group containing at least one of vinyl, acrylic, amino, mercapto, or vinyl chloride functional group; and
R² is a lower alkyl group;
(B) acid component comprising a member selected from the group consisting of water-soluble organic acids, H₃BO₃ and H₃PO₃; and
(D) water.

VII. An aqueous coating composition formed by mixing
(A) at least one silane of formula (1)
   R¹ₙSi(OR²)₄₋ₙ (1)
wherein R¹ represents lower alkyl, C₆-C₈ aryl, or a functional group containing at least one of vinyl, acrylic, amino, mercapto, or vinyl chloride functional group; and
R² is a lower alkyl group;
(C) alkali component; and
(D) water.

VIII. An aqueous coating composition formed by combining
(A) at least one silane of the formula (1)
   R¹Si(OR²)₃ (1)
wherein
R¹ represents lower alkyl group, C₆-C₈ aryl group or a bifunctional silane containing vinyl, acrylic, amino, or vinyl chloride functional group; and
R² is a lower alkyl group;
(E) (ii) colloidal aluminum hydroxide, (iii) metal alcoholate of the formula (3)
   M(OR³)ₘ (3)
wherein
M is a metal of valence m,
R³ is a lower alkyl group,
m is an integer of 3 or 4,
or (iii) mixture of (ii) and (iii); and
(D) water.

IX. An aqueous coating composition formed by mixing
(A) at least one silane of the formula (1)
   R¹Si(OR²)₃ (1)
wherein
R¹ represents lower alkyl group, C₆-C₈ aryl group or a bifunctional silane containing vinyl, acrylic, amino, or vinyl chloride functional group; and
R² is a lower alkyl group;
(D) water;
(G) chromium acetate hydroxide; and
(H) lower alkanol.

X. an aqueous coating composition formed by combining
(A) at least one silane of the formula (1)
   R¹Si(OR²)₃ (1)
wherein
R¹ represents lower alkyl group, C₆-C₈ aryl group or a functional group including at least one of vinyl, acrylic, amino, mercapto, or vinyl chloride functional group; and
R² is a lower alkyl group;
(D) water;
(E) (ii) colloidal aluminum hydroxide, (iii) metal alcoholate of the formula (3)
   M(OR³)ₘ (3)
wherein
M is a metal of valence m,
R³ is a lower alkyl group,
m is an integer of 3 or 4,
or (iii) mixture of (ii) and (iii);
(F) alkali metal silicate, which may be hydrolyzed; and
(H) lower alkanol.

XI. a non-metallic aqueous coating composition formed by mixing
(A) at least one silane of the formula (1)
   R¹Si(OR²)₃ (1)
wherein
R¹ represents lower alkyl group, C₆-C₈ aryl group or a functional group including at least one of vinyl, acrylic, amino, mercapto, or vinyl chloride functional group; and

R² is a lower alkyl group;
(A″) 3-(2-aminoethylamino)propyltrimethoxysilane or 3-aminopropyltrimethoxysilane;
(D) water;
(H) lower alkanol; and
(I) epoxide silane.

XII. an aqueous coating composition formed by mixing
(A) at least one silane of the formula (1)
   R¹Si(OR²) (1)
wherein
R¹ represents lower alkyl group, C₆-C₈ aryl group or a functional group including at least one of vinyl, acrylic, amino, mercapto, or vinyl chloride functional group; and
R² is a lower alkyl group;
(B) boric acid;
(C) at least one alkali component comprising an hydroxide or carbonate of divalent metal;
(D) water;
(H) lower alkanol, and
(J) ethyl polysiloxane.

In addition to silane compound(s) of formula (1), the composition may additionally include one or more bistrifunctional silane(s), such as represented by the following formula (2):
X[R¹Si(OR²)₃]₂ (2)
where R¹ and R² are as defined above, and X represents an amino group (—NH) or keto group, as a basic catalyst, not requiring acid stabilization. Representative example of aminosilane or ketosilane catalyst according to formula (2), include, for example, bis(trimethoxypropylsilane)amine, bis(trimethoxyethylsilane)amine, di(trimethoxybutylsilane) ketone, di(trimethoxypropylsilane) ketone, and the like. The silane compounds of formula (4) function as a less active basic catalyst, not requiring acidic passivation. Minor amounts, usually from about 1 to about 10 parts, preferably, from about 2 to about 8 parts, of compound of formula (4) per 100 parts of silane compound(s) of formula (1) provide satisfactory results.

The compositions of this embodiment may further include one or more additional additives for functional and/or esthetics effects, such as, for example, (d) colloidal aluminum hydroxide, (e) metal alcoholate, (f) silica and/or silicates, (g) color forming silanol condensation catalyst, (h) epoxide silane, (i) ultraviolet absorber, (j) water, (k) co-solvent, and others, as described above.

The above-noted optional ingredients may be used singly or in any combination in the coating composition of this invention.

Within the above general proportions, and based on the weight of the entire composition, the amount (parts by weight) of the individual classes of ingredients, will usually fall within the following ranges: silane component (a) from about 15 to about 25 parts, preferably, as a mixture of from about 15 to about 20 parts of methyltrimethoxysilane and from about 1 to about 5 parts of phenyltrimethoxysilane; base component condensation catalyst (b), when present, from about 0.1 to 3 parts, preferably from about 0.2 to 2.5 parts; acid component condensation catalyst (b), when present, from about 0.2 to about 0.8 part; solvent, e.g., isopropyl alcohol, to provide the appropriate viscosity, generally, from about 5 to about 60 parts, preferably, from about 10 to about 40 parts; water (j), when present, from about 2.5 parts by weight to about 40 parts; silicate component (f), when used, from 0 to about 15 parts by weight; mono-lower alkyl ether of ethylene glycol and/or other co-solvent (k), when used, from 0 to about 3 parts; ultra-violet light absorber (i), when used, from 0 to about 2 parts by weight; colloidal aluminum hydroxide and/or the metal alcoholate, when used, from 0 to about 0.5 part by weight.

Furthermore, the above general and preferred amounts of the respective ingredients apply equally to the various embodiments I-XII, of the coating compositions, as identified above.

If the activity of the coating compositions results, when applied too thickly, in a random distribution of lumps, presumably due to gelling, the coating compositions preferably are applied in the minimum amount necessary to substantially completely coat the surface to be protected. Coating thicknesses of less than 1 milimeter (mm), preferably, less than about 0.5 milimeter (mm), are usually satisfactory.

While not wishing to be bound by any particular theory, it is believed that the effectiveness of the instant classes of silane/siloxane coating compositions arises from the ability of such coatings to form dendritic interfacial linkages that effect their performance in thin layers, normally about 5 to about 150 millionths of an inch.

The coated substrates of medical articles by virtue of the chemical bonding and silica or siloxane bonding, with the additional dendritic linkages, producing a glass-like structural formation over the chemical bond area and reduces the available chemical activity on the coated surfaces.

In addition, in view of the hydrophobic nature of the applied coatings, the coated surfaces will stay cleaner and sterile for longer periods of time.

It is believed that, the coated tissue or articles of the present invention do not promote growth of microorganisms, e.g., fungi, viruses, mold spores, yeast, bacteria, and the like. In the case of medical articles, for instance, since growth of microorganisms is inhibited, when a medical article is coated as described above, subsequent introduction of microorganisms into a patient is greatly inhibited or prevented.

While this invention has been particularly shown and described with reference to preferred embodiments thereof, it will be understood by those skilled in the art that various changes in form and details may be made therein without departing from the scope of the invention encompassed by the appended claims.

1. A method for forming an antibacterial coating on live tissue or a medical article comprising:
a) applying to a live tissue region or a medical article surface a siloxanol polymer composition produced by combining:
i) an acidic aqueous component including divalent metal cations; and
ii) a non-aqueous component including a silane having a formula RSi(OR¹)₃, wherein R is a radical selected from the group consisting of lower alkyl, vinyl, phenyl, 3,3,3-trifluoropropyl, γ-glycidyloxypropyl, and γ-methacryloxypropyl, and R¹ is a hydrolyzable hydrocarbyl group, at least about 70 percent by weight of the silane being CH₃Si(OCH₃)₃; and
b) curing the siloxanol polymer, thereby forming the antimicrobial coating.

2. A method for forming an antimicrobial coating on live tissue or a medical article comprising:
a) combining an acidic aqueous dispersion of divalent metal cation selected from the group consisting of Ca⁺², Mn⁺², Zn⁺², and Cu⁺², said dispersion containing sufficient acid to provide a pH of from about 2.2 to about 2.8 with a non-aqueous composition comprising at least one trialkoxy silane of the formula RSi(OR¹)₃, wherein R is a radical selected from the group consisting of lower alkyl, vinyl, phenyl, 3,3,3-trifluoropropyl, γ-glycidyloxypropyl, and γ-methacryloxypropyl, and R¹ is a hydrolyzable hydrocarbyl group, at least about 70 percent by weight of the silane being CH₃Si(OCH₃)₃;
b) allowing the resulting mixture to stand until the pH increases to at least about 2.5, whereby the silane is hydrolyzed to silanol and the silanol is partially condensed to a siloxanol polymer; whereby the amount of divalent metal cation in the acidic aqueous dispersion is from about 1.2 to about 2.4 millimoles, per molar equivalent of the silane sesquioxide, calculated as methyl silane sesquioxide, and further wherein at least one of the aqueous dispersion and the non-aqueous composition further comprises a volatile organic solvent;
c) applying the mixture including the siloxanol polymer to a live tissue region or surface of a medical article; and
d) curing the siloxanol polymer to form silane sesquioxide, thereby forming the antimicrobial coating,
wherein the divalent metal cations are present in the aqueous dispersion in an amount of from about 1.2 millimoles to about 2.4 millimoles, per molar equivalent of the partial condensate, calculated as methyl silane sesquioxide.

3. A medical article comprising an antimicrobial coating that is produced by curing a partial condensate of a silanol of the formula RSi(OH)₃, wherein R is selected from the group consisting of lower alkyl, vinyl, phenyl, 3,3,3-trifluoroproppyl, γ-glycidyloxypropyl, and γ-methacryloxypropyl and wherein at least about 70 percent by weight of the silanol is CH₃Si(OH)₃, and the partial condensate of the silanol is present in an acidic composition that includes an aqueous-organic solvent and divalent metal cations.

4. The medical article of claim 3, wherein the partial condensate is formed by hydrolyzing a silane of the formula RSi(OR¹)₃, wherein R¹ is a hydrolyzable hydrocarbyl group, at least about 70 percent by weight of the silane being CH₃Si(OCH₃)₃;

5. A medical article comprising an antimicrobial coating that is produced by curing a partial condensate of a silanol of the formula RSi(OH)₃, wherein R is selected from the group consisting of lower alkyl, vinyl, phenyl, 3,3,3-trifluoroproppyl, γ-glycidyloxypropyl, and γ-methacryloxypropyl and wherein at least about 70 percent by weight of the silanol is CH₃Si(OH)₃, and wherein the partial condensate of the silanol is present in an acidic composition that includes an aqueous-organic solvent and colloidal silica.

6. A method for forming an antimicrobial coating on live tissue or a medical article, comprising:
a) applying to a live tissue region or a medical article surface an aqueous coating composition comprising a dispersion of divalent metal cations in lower aliphatic alcohol-water solution of the partial condensate of at least one silanol of the formula RSi(OH)₃, wherein R is a radical selected from the group consisting of lower alkyl, vinyl, phenyl, 3,3,3-trifluoropropyl, γ-glycidyloxypropyl, and γ-methacryloxypropyl, at least about 70 percent by weight of the silanol being CH₃Si(OH)₃, acid in amount to provide a pH in the range of from about 2.5 to about 6.2, and said divalent metal cations in an amount of from about 1.2 millimoles to about 2.4 millimoles, per molar equivalent of the partial condensate, calculated as methyl silane sesquioxide; and
b) allowing the aliphatic alcohol and water of the aqueous coating composition to evaporate and the partial condensate to cure to the corresponding silane sesquioxide, thereby forming the antimicrobial coating.

7. A medical article comprising an antimicrobial coating that is formed by reacting:
a) a silane component of the formula R¹Si(OR²)₃, wherein, R¹ is a lower alkyl group, a phenyl group or a vinyl, an acrylic, an amino, a mercapto, or a a vinyl chloride functional group; and each R² is independently a lower alkyl group;
b) at least one of:
i) a base component selected from the group consisting of a metal hydroxide, an aminosilane and a ketosilane; or
ii) an acid component selected from the group consisting of a water-soluble organic acid, boric acid and phosphorous acid or salt thereof; and
c) water.

8. A method for forming an antimicrobial coating on live tissue or a medical article comprising:
a) applying to a live tissue region or a surface of a medical article a mixture formed by combining at least one silane of the formula R¹Si(OR²)₃, wherein R¹ is a lower alkyl group, a phenyl group or a bifunctional silane containing vinyl, acrylic, amino, or vinyl chloride functional group and R² is a lower alkyl group with at least one component selected from the group consisting of colloidal aluminum hydroxide and a metal alcoholate of the formula M(OR³)ₘ, wherein M is a metal of valence m, R³ is a lower alkyl group, m is an integer of 3 or 4, and water; and
b) curing the mixture, thereby forming the antimicrobial coating.

9. A method for forming an antimicrobial coating on live tissue or a medical article, comprising:
a) applying to a live tissue region or a surface of a medical article a non-metallic aqueous coating composition formed by combining:
i) at least one silane of the formula R¹Si(OR²)₃, wherein R¹ is a lower alkyl group, a phenyl group or a functional group including at least one of vinyl, acrylic, amino, mercapto, or vinyl chloride functional group and R² is a lower alkyl group;
ii) (3-(2-aminoethylamino)propyltrimethoxysilane or 3-aminopropyltrimethoxysilane;
iii) water;
iv) epoxide silane;
v) lower alkanol; and
b) curing the coating composition, thereby forming the antimicrobial coating.

10. A method for forming an antimicrobial coating on live tissue or a medical article, comprising:
a) applying to a live tissue region or a surface of a medical article a non-metallic aqueous coating composition formed by combining:
i) at least one silane of the formula R¹Si(OR²)₃ wherein R¹ is a lower alkyl group, a phenyl group or a functional group including at least one of vinyl, acrylic, amino, mercapto, or vinyl chloride functional group; and R² is a lower alkyl group;
ii) at least one alkali component comprising an hydroxide or carbonate of divalent metal;
iii) boric acid;
iv) water;
v) ethyl polysiloxane; and
vi) lower alkanol; and
b) curing the coating composition, thereby forming the antimicrobial coating.

11. A method for forming an antimicrobial coating on live tissue or a medical article comprising:
a) applying to a live tissue region or a surface of a medical article a composition obtained by combining:
i) at least two silanes represented, independently, by formula R¹Si(OR²)₃ wherein at least one R¹ represents a lower alkyl group in at least one silane of formula, at least one R¹ represents a group containing a functional mercapto group, and any other R¹ groups may represent a phenyl group, or a functional group including at least one of vinyl, acrylic, amino, or vinyl chloride functional group; and R² represents a lower alkyl group; and
ii) lower alkanol; and
b) curing the coating composition, thereby forming the antimicrobial coating.

12. A method for forming an antimicrobial coating on live tissue or a medical article comprising:
a) applying to a live tissue region or a surface of a medical article a composition, which in combination with water, forms a neutral or basic composition, wherein the composition comprises the product obtained by combining in a lower alkanol solvent, at least two silanes, represented independently by formula R¹Si(OR¹)₃ wherein at least one R¹ represents lower alkyl group, at least one R¹ represents a group, containing a functional amino group, any other R¹ group represents, phenyl, or a functional group including at least one of vinyl, acrylic, or vinyl chloride functional group, R² represents lower alkyl group; and
b) curing the composition, thereby forming the antimicrobial coating on the medical article.

13. A method for coating live tissue or a medical article, comprising:
a) applying to a live tissue region or a surface of a medical article a non-metallic non-aqueous coating composition formed by combining:
i) at least one silane of formula R¹ₙSi(OR²)₄₋ₙ, wherein R¹ represents a lower alkyl group, an aryl group or a functional group containing at least one of vinyl, acrylic, amino, mercapto, or vinyl chloride functional groups; R² represents a lower alkyl group; and, n is a number of 1 to 2; and
ii) at least one compound selected from the group consisting of vinyltriacetoxysilane, colloidal aluminum hydroxide and at least one metal alcoholate of formula M(OR³)ₘ wherein M represents a metal of valence m, R3 represents a lower alkyl group, and m is a number of 2 to 4; and
b) curing the coating composition, thereby forming the antimicrobial coating.

14. A method for inhibiting or preventing microbial infections comprising:
a) applying to live tissue or to a medical article substrate a siloxanol polymer composition produced by combining:
i) an acidic aqueous component including divalent metal cations; and
ii) a non-aqueous component including a silane having a formula RSi(OR¹)₃, wherein R is a radical selected from the group consisting of lower alkyl, vinyl, phenyl, 3,3,3-trifluoropropyl, γ-glycidyloxypropyl, and γ-methacryloxypropyl, and R¹ is a hydrolyzable hydrocarbyl group, at least about 70 percent by weight of the silane being CH₃Si(OCH₃)₃; and
b) curing the siloxanol polymer, thereby forming on the live tissue or the medical article substrate a coating that inhibits or prevents colonization or growth of microorganisms.

15. A method for inhibiting or preventing microbial infections comprising:
a) combining an acidic aqueous dispersion of divalent metal cation selected from the group consisting of Ca⁺², Mn⁺², Zn⁺², and Cu⁺², said dispersion containing sufficient acid to provide a pH of from about 2.2 to about 2.8 with a non-aqueous composition comprising at least one trialkoxy silane of the formula RSi(OR¹)₃, wherein R is a radical selected from the group consisting of lower alkyl, vinyl, phenyl, 3,3,3-trifluoropropyl, γ-glycidyloxypropyl, and γ-methacryloxypropyl, and R¹ is a hydrolyzable hydrocarbyl group, at least about 70 percent by weight of the silane being CH₃Si(OCH₃)₃;
b) allowing the resulting mixture to stand until the pH increases to at least about 2.5, whereby the silane is hydrolyzed to silanol and the silanol is partially condensed to a siloxanol polymer; whereby the amount of divalent metal cation in the acidic aqueous dispersion is from about 1.2 to about 2.4 millimoles, per molar equivalent of the silane sesquioxide, calculated as methyl silane sesquioxide, and further wherein at least one of the aqueous dispersion and the non-aqueous composition further comprises a volatile organic solvent;
c) applying the mixture including the siloxanol polymer to live tissue or a medical article substrate; and
d) curing the siloxanol polymer to form silane sesquioxide, thereby forming on the live tissue or the medical article substrate a coating that inhibits or prevents colonization or growth of microorganisms,
wherein the divalent metal cations are present in the aqueous dispersion in an amount of from about 1.2 millimoles to about 2.4 millimoles, per molar equivalent of the partial condensate, calculated as methyl silane sesquioxide.

16. A method for inhibiting or preventing microbial infections comprising:
a) applying to live tissue or a medical article substrate an aqueous coating composition comprising a dispersion of divalent metal cations in lower aliphatic alcohol-water solution of the partial condensate of at least one silanol of the formula RSi(OH)₃, wherein R is a radical selected from the group consisting of lower alkyl, vinyl, phenyl, 3,3,3-trifluoropropyl, γ-glycidyloxypropyl, and γ-methacryloxypropyl, at least about 70 percent by weight of the silanol being CH₃Si(OH)₃, acid in amount to provide a pH in the range of from about 2.5 to about 6.2, and said divalent metal cations in an amount of from about 1.2 millimoles to about 2.4 millimoles, per molar equivalent of the partial condensate, calculated as methyl silane sesquioxide; and
b) allowing the aliphatic alcohol and water of the aqueous coating composition to evaporate and the partial condensate to cure to the corresponding silane sesquioxide, thereby forming on the live tissue or the medical article substrate a coating that inhibits or prevents colonization or growth of microorganisms.

17. A method for inhibiting or preventing microbial infections comprising:
a) applying to a live tissue or a medical article substrate a mixture formed by combining at least one silane of the formula R¹Si(OR²)₃, wherein R¹ is a lower alkyl group, a phenyl group or a bifunctional silane containing vinyl, acrylic, amino, or vinyl chloride functional group and R² is a lower alkyl group with at least one component selected from the group consisting of colloidal aluminum hydroxide and a metal alcoholate of the formula M(OR³)ₘ, wherein M is a metal of valence m, R³ is a lower alkyl group, m is an integer of 3 or 4, and water; and
b) curing the mixture, thereby forming on the live tissue or the medical article substrate a coating that inhibits or prevents colonization or growth of microorganisms.

18. A method for inhibiting or preventing microbial infections comprising:
a) applying to live tissue or a medical article substrate a non-metallic aqueous coating composition formed by combining:
i) at least one silane of the formula R¹Si(OR²)₃, wherein R¹ is a lower alkyl group, a phenyl group or a functional group including at least one of vinyl, acrylic, amino, mercapto, or vinyl chloride functional group and R² is a lower alkyl group;
ii) (3-(2-aminoethylamino)propyltrimethoxysilane or 3-aminopropyltrimethoxysilane;
iii) water;
iv) epoxide silane;
v) lower alkanol; and
b) curing the coating composition, thereby forming on the live tissue or the medical article substrate a coating that inhibits or prevents colonization or growth of microorganisms.

19. A method for inhibiting or preventing microbial infections comprising:
a) applying to live tissue or a medical article substrate a non-metallic aqueous coating composition formed by combining:
i) at least one silane of the formula R¹Si(OR²)₃ wherein R¹ is a lower alkyl group, a phenyl group or a functional group including at least one of vinyl, acrylic, amino, mercapto, or vinyl chloride functional group; and R² is a lower alkyl group;
ii) at least one alkali component comprising an hydroxide or carbonate of divalent metal;
iii) boric acid;
iv) water;
v) ethyl polysiloxane; and
vi) lower alkanol.
b) curing the coating composition, thereby forming on the live tissue or the medical article substrate a coating that inhibits or prevents colonization or growth of microorganisms.

20. A method for inhibiting or preventing microbial infections comprising:
a) applying to live tissue or a medical article substrate a composition obtained by combining:
i) at least two silanes represented, independently, by formula R¹Si(OR²)₃ wherein at least one R¹ represents a lower alkyl group in at least one silane of formula, at least one R¹ represents a group containing a functional mercapto group, and any other R¹ groups may represent a phenyl group, or a functional group including at least one of vinyl, acrylic, amino, or vinyl chloride functional group; and R² represents a lower alkyl group; and
ii) lower alkanol; and
b) curing the coating composition, thereby forming on the live tissue or the medical article substrate a coating that inhibits or prevents colonization or growth of microorganisms.

21. A method for inhibiting or preventing microbial infections comprising:
a) applying to live tissue or a medical article substrate a composition which in combination with water, forms a neutral or basic composition, wherein the composition comprises the product obtained by combining in a lower alkanol solvent, at least two silanes, represented independently by formula R¹Si(OR¹)₃ wherein at least one R¹ represents lower alkyl group, at least one R¹ represents a group, containing a functional amino group, any other R¹ group represents, phenyl, or a functional group including at least one of vinyl, acrylic, or vinyl chloride functional group, R² represents lower alkyl group; and
b) curing the composition, thereby forming on the live tissue or the medical article substrate a coating that inhibits or prevents colonization or growth of microorganisms.

22. A method for inhibiting or preventing microbial infections comprising:
a) applying to live tissue or a medical article substrate a non-metallic non-aqueous coating composition formed by combining:
i) at least one silane of formula R¹ₙSi(OR²)₄₋ₙ wherein R¹ represents a lower alkyl group, an aryl group or a functional group containing at least one of vinyl, acrylic, amino, mercapto, or vinyl chloride functional groups; R² represents a lower alkyl group; and, n is a number of 1 to 2; and
ii) at least one compound selected from the group consisting of vinyltriacetoxysilane, colloidal aluminum hydroxide and at least one metal alcoholate of formula M(OR³)ₘ wherein M represents a metal of valence m, R3 represents a lower alkyl group, and m is a number of 2 to 4; and
b) curing the coating composition, thereby forming on the live tissue or the medical article substrate a coating that inhibits or prevents colonization or growth of microorganisms.
